(19) United States
(12) Patent Application Publication
(10) Pub. No.: US 2005/0064472 A1
(43) Pub. Date: Mar. 24, 2005

(54) Methods of monitoring gene expression

(75) Inventors: Mamatha Shekar, Cupertino, CA (US); Liu Chunmei, Cupertino, CA (US); Janet A. Warrington, Los Altos, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/896,157

(22) Filed: Jul. 20, 2004

Related U.S. Application Data
(60) Provisional application No. 60/489,651, filed on Jul. 23, 2003.

(51) Int. Cl. C12Q 1/68; C12P 19/34

(52) U.S. Cl. 435/6; 435/91.2

(57) ABSTRACT

The disclosed methods relate to the use of an amplification product generated from RNA to validate an expression measurement obtained by microarray analysis. RNA may be amplified as cRNA or cDNA and an aliquot of the amplified sample, cRNA or cDNA, may be analyzed for the amount of selected RNAs using an analysis method such as quantitative RT-PCR. Microarry measurements for selected RNAs may be compared to measurements obtained for the same RNAs using an independent analysis method.

### BACKGROUND

Many cellular events and processes are characterized by altered expression levels of one or more genes. Differences in gene expression correlate with many physiological processes such as cell cycle progression, cell differentiation and cell death. Changes in gene expression patterns also correlate with changes in disease or pharmacological state. For example, the lack of sufficient expression of functional tumor suppressor genes and/or the over expression of oncogene/protooncogenes could lead to tumorgenesis (Marshall, Cell, 64: 313-326 (1991); Weinberg, Science, 254: 1138-1146 (1991), incorporated herein by reference for all purposes). Thus, changes in the expression levels of particular genes (e.g. oncogenes or tumor suppressors) serve as signposts for different physiological, pharmacological and disease states.

Most biological processes involve large-scale changes in gene expression levels and/or patterns. The advent of microarray technology has made it possible to study these changes in expression in order to identify new complex phenotypic markers or to identify genes involved in particular cellular processes. Gene expression profiles produce a snapshot that reflects the biological status of the sample, but in many circumstances biological status will reflect more than one characteristic of the sample. For example, when comparing tumor samples from two patients, there will be changes that correlate with differences between the states of the tumors as well as changes that correlate with the different physiological states of the two patients. Currently, array technology is most useful in establishing broad patterns of gene expression and in screening for differential gene expression.

### SUMMARY OF THE INVENTION

A method for validating the results of a microarray analysis of an experimental nucleic acid sample by quantifying a selected species of RNA from the nucleic acid sample is disclosed. In one embodiment the method involves isolating total RNA from the experimental sample, isolating polyA RNA from the total RNA and amplifying the poly (A) RNA. The poly (A) RNA may be amplified as cRNA or cDNA depending on the method of amplification. The cDNA may be double or single stranded or a mixture of double and single stranded.

In one embodiment cRNA is generated from the polyA RNA. The cRNA is used for quantifying the selected species of RNA using a method selected from the following methods: quantitative reverse transcription-polymerase chain reaction (QRT-PCR), Northern blot hybridization and RNase protection analysis. The results obtained are compared to the results obtained from the microarray analysis for the selected RNA.

In some embodiments cRNA is generated from polyA RNA by mixing polyA RNA with a primer comprising an RNA polymerase promoter sequence and a region of poly(dT) wherein the region of poly(dT) is 3′ of the RNA polymerase promoter sequence, extending the primer using reverse transcriptase to generate first strand cDNA, generating second strand cDNA resulting in double stranded cDNA comprising an RNA polymerase promoter, and generating cRNA from the double stranded cDNA using an RNA polymerase that recognizes the RNA polymerase promoter. The RNA polymerase may be, for example, T7, T3 or SP6. The cRNA may be labeled during synthesis or by end labeling. The label may be fluorescent or chemiluminescent. In one embodiment the label is biotin. The cRNA may be fragmented before being used for quantitation.

In some embodiments the poly (A) RNA is amplified by reverse transcription using random primers. The resulting cDNA may be fragmented, labeled and hybridized to an array. An aliquot of cDNA may be analyzed by, for example, QRT-PCR, for selected messages. In some embodiments the cDNA is further amplified before analysis.

In some embodiments the amount of cRNA used for quantitation is approximately equal to 1, 2, 5, or 10% of the amount of total RNA isolated. Any RNA species may be analyzed, for example, GAPDH, Nexin or calreticulin. In preferred embodiments a plurality of species are analyzed by both microarray analysis and a second analysis method. The measurements are compared to determine the difference between the two analysis methods for a selected RNA species. Preferably the two analysis methods vary by less than 50%, 25%, 5%, 1%, or 0.1%.

In some embodiments the total RNA sample comes from an experimental sample that is available in a limiting amount, for example, a fine needle aspirate or from laser capture microdissection (LCM).

### BRIEF DESCRIPTION OF THE FIGURES

FIG. 1 shows a schematic of one embodiment. Microarray analysis of the sample involves isolation of polyA RNA which is then used to make labeled cRNA. The labeled cRNA is hybridized to an array and the hybridization pattern is analyzed. For validation aliquots of RNA may be used from the total RNA or from the cRNA.

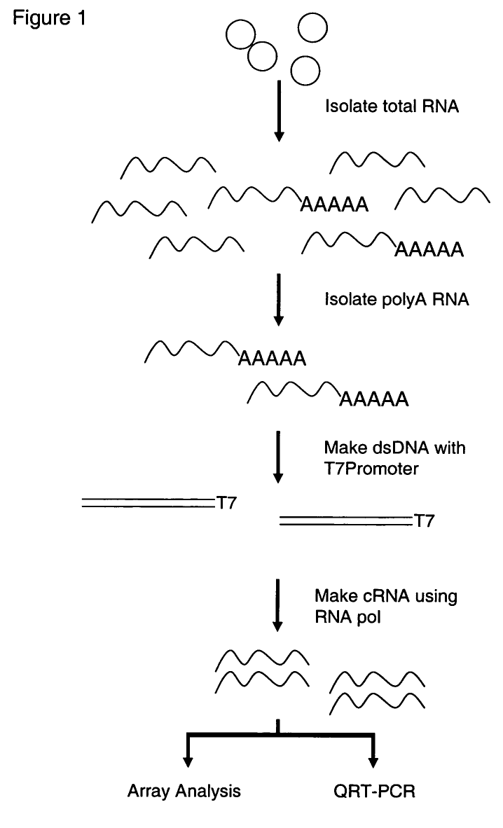

### DETAILED DESCRIPTION

a) General

The present invention has many preferred embodiments and relies on many patents, applications and other references for details known to those of the art. Therefore, when a patent, patent application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. Nos. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

b) Definitions

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “cartridge” as used herein refers to a body forming an area or space referred to as a well wherein a microarray is contained and separated from the passage of liquids.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “differentially expressed” as used herein means that the measurement of a cellular constituent varies in two or more samples. The cellular constituent can be either upregulated in the experimental relative to the reference or down-regulated in the experimental relative to the reference. Differential gene expression can also be used to distinguish between cell types or nucleic acids. See U.S. Pat. No. 5,800,992.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

The term “hybridization conditions” as used herein will typically include salt concentrations of less than about 1 M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Hybridizations are usually performed under stringent conditions, for example, at a salt concentration of no more than about 1 M and a temperature of at least 25° C., preferably between 40 and 55° C., and more preferably between 44 and 50° C. The pH of the hybridization buffer is preferably between 6 and 8 and more preferably between 6.4 and 7. A buffer such as MES, HEPES, MOPS, or Tris, for example, is preferably included. A chelator, such as EDTA may be included, for example at about 5 to 100 mM, preferably at about 20 to 30 mM. A detergent may also be included, for example, about 0.01% Tween 20. Other reagents that may be included in the hybridization include, for example, DMSO, herring sperm DNA, Cot-1 DNA and tetramethyl ammonium chloride which may be present at a concentration of between 2 and 3 M.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “isolated nucleic acid” as used herein mean an object species that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “microtiter plates” as used herein refers to arrays of discrete wells that come in standard formats (96, 384 and 1536 wells) which are used for examination of the physical, chemical or biological characteristics of a quantity of samples in parallel.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

One measurement of cellular constituents that is particularly useful in the present invention is the expression profile. As used herein, an “expression profile” comprises measurement of the relative abundance of a plurality of cellular constituents. Such measurements may include, for example, RNA or protein abundances or activity levels. The expression profile can be a measurement for example of the transcriptional state or the translational state. See U.S. Pat. Nos. 6,040,138, 5,800,992, 6,020,135, 6,033,860 and U.S. Ser. No. 09/341,302 which are hereby incorporated by reference in their entireties.

The “transcriptional state” of a sample includes the identities and relative abundances of a plurality of the RNA species present in the sample, especially mRNAs present in the sample. Preferably, a substantial fraction of all constituent RNA species in the sample are measured, but at least, a sufficient fraction is measured to characterize the state of the sample. The transcriptional state is an aspect of the biological state. It can be conveniently determined by measuring transcript abundances by any of several existing gene expression technologies.

The “translational state” includes the identities and relative abundances of a plurality of the protein species in the sample. As is known to those of skill in the art, the transcriptional state and translational state are related.

Expression may be analyzed with a gene expression monitoring system. Several such systems are known. See, e.g., U.S. Pat. No. 5,677,195; Wodicka et al., Nature Biotech. 15:1359-1367(1997); and Lockhart et al., Nature Biotech. 14:1675-1680 (1996). A gene expression monitoring system according to the present invention may be a nucleic acid probe array such as the GeneChip® nucleic acid probe array (Affymetrix, Santa Clara, Calif.). A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, nylon, polystyrene, polycarbonate, plastics, glass, ceramic, stainless steel, or the like. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded.

The gene expression monitoring system, in a preferred embodiment, may comprise a nucleic acid probe array (such as those described above), membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue.

Use of cRNA for Validation of Microarray Results

Gene expression profiling using high density oligonucleotide arrays is a powerful approach to characterization of expression. Microarrays may be used to determine the expression levels of a collection of genes simultaneously in an experimental sample. Array technology may be used, for example, in establishing broad patterns of gene expression and in screening for differential gene expression. Gene expression levels may be determined, for example, relative to other genes in the sample or relative to the same gene from a different sample. Having experimentally determined a gene expression level for a gene in a sample it is often desirable to confirm that result using a second independent assay method. Confirmation of the gene expression levels for one or more genes from a sample may be used to validate the assay method, for example, if an array is used to determine the gene expression levels for 10,000 genes in parallel, a second assay method may be used to determine the expression levels for a subset of the genes, for example, 5 to 10 genes or 10 to 50 genes or more. Agreement as to the expression levels obtained for the subset of genes between the two assay methods validates the accuracy of the array method.

Microarray analysis may be performed using a relatively large amount of starting material, for example 5 to 10 μg of total RNA but frequently only smaller amounts of RNA are available, for example when the number of cells of interest are limiting as with cell sorting, primary cell culture, embryonic dissection, fine needle aspirates or laser capture microdissection. With amplification methods as little as 10 ng total RNA may be sufficient for global expression analysis. When amounts of total RNA available are very small it may be preferable to use the entire total RNA sample for amplification and use amplified sample for confirmation of microarray results.

A number of widely used procedures exist for validation of a microarray based measurement of the expression pattern of a particular mRNA across RNA samples. Typically these methods require the researcher to use an aliquot of the mixed population of RNA obtained from the sample for use with the validation method, meaning less RNA to be amplified to prepare labeled nucleic acid to hybridize to the array and generally less material to hybridize to the array, possibly impacting the sensitivity of the analysis. In a preferred embodiment the entire mixed population of RNA obtained from the sample is amplified and an aliquot of the amplified sample is used for secondary analysis. In this way the impact of the removal of material for secondary analysis is minimized. For examples of microarray analysis and methods of providing controls for microarray analysis see, for example, Stamey et al. J. Urol 170:2263-8, (2003), Chen et al. J.Urol 169:1316-9, (2003) and Luzzi et al. J Mol Diagn 5:9-14 (2003).

Commonly used methods of secondary validation include Northern blot hybridization, nuclease protection assays (NPA), in situ hybridization, RNase protection assay, and reverse transcription polymerase chain reaction (RT-PCR) or quantitative RT-PCR (QRT-PCR). QRT-PCR is the most sensitive of these techniques and may be used to detect a single copy of mRNA from a gene. In RT-PCR an RNA template is copied into a complementary DNA transcript (cDNA) using reverse transcriptase. The cDNA sequence of interest is then amplified exponentially using PCR. This technique can be used for relative or absolute quantitation of transcripts.

Methods for monitoring gene expression of a plurality of genes in parallel are disclosed. In many embodiments RNA expression products are amplified to generate an amplified nucleic acid sample that is representative of the transcriptional state of the experimental sample. The amplified sample is analyzed by hybridization to a microarray that contains probes for thousands of different transcripts and the hybridization pattern is analyzed to obtain measurements for the expression levels of a plurality of genes, for example, for more than 500 or more than 1,000 genes. The amplified sample is also analyzed using a second, preferably independent, method of analysis to obtain a second measurement for the expression of at least one of the genes for which a measurement was obtained by the analysis of the microarray hybridization analysis. The second measurement is compared to the microarray measurement for the same transcript to determine the agreement between the two measurements. Preferably the measurements differ by less than 50%, more preferably by less than 25%, more preferably by less than 10% and most preferably by less than 5%.

In Northern blotting RNA samples are first separated by size by agarose gel electrophoresis under denaturing conditions. The RNA is then transferred to a membrane, crosslinked and hybridized with a labeled probe. Probes may be labeled by any method, including random-primed, nick-translated, or PCR-generated DNA probes, in vitro transcribed RNA probes, and oligonucleotides. Additionally, sequences with only partial homology may be used as probes.

Nuclease Protection Assay (NPA) (including both ribonuclease protection assays and S1 nuclease assays) is an extremely sensitive method for the detection and quantitation of specific mRNAs. An probe that is complementary to the RNA is labeled (radiolabeled or nonisotopic) and hybridized to an RNA sample in solution. After hybridization, single-stranded, unhybridized probe and RNA are degraded by nucleases. The remaining protected fragments are separated on an acrylamide gel. RNase H, for example, digests RNA DNA hybrids but does not digest RNA or DNA that is not part of a RNA-DNA duplex.

RT-PCR is a sensitive technique for mRNA detection and quantitation. The two steps of the process are: synthesis of cDNA from RNA by reverse transcription (RT) and amplification of a specific cDNA by polymerase chain reaction (PCR), preferably using a pair of primers specific for the RNA to be detected. In a preferred embodiment the primers used for the PCR step of RT-PCR are selected to amplify only a few hundred bases rather than the complete mRNA sequence.

The RNA in one embodiment is high quality and free from genomic DNA contamination. But degraded RNA samples may be used because RT-PCR typically begins with a reverse transcriptase reaction. The RT reaction uses an RNA template, for example, total RNA, polyA RNA or cRNA as presently disclosed, a primer, rNTPs, buffer and a reverse transcriptase (M-MLV or AMV RT for example).

The RT reaction may, for example, be incubated for 1 hour at 42° C. to generate a single-stranded DNA molecule complementary to the RNA (cDNA). The cDNA serves as a template in the PCR reaction. Components of the PCR, in addition to the cDNA, include dNTPs, buffer, thermostable DNA polymerase and primers specific for the gene of interest (gene specific primers). The cDNA is amplified exponentially using cycles of denaturation, annealing and extension. Because amplification is exponential, small sample-to-sample concentration and loading differences are amplified as well; therefore, PCR requires careful optimization when used for quantitative mRNA analysis.

Like other methods of mRNA analysis, such as Northern blots and nuclease protection assays, RT-PCR can be used for relative or absolute quantitation.

Relative quantitation compares transcript abundance across multiple samples, using a co-amplified internal control for sample normalization. Results are expressed as ratios of the gene specific signal to the internal control signal. This yields a corrected relative value for the gene specific product in each sample. These values may be compared between samples for an estimate of the relative expression of target RNA in the samples.

Absolute quantitation, using competitive RT-PCR, measures the absolute amount (e.g., 2.1×10⁴ copies) of a specific mRNA sequence in a sample. Dilutions of a synthetic RNA (identical in sequence, but slightly shorter than the endogenous target) are added to sample RNA replicates and are co-amplified with the endogenous target. The PCR product from the endogenous transcript is then compared to the concentration curve created by the synthetic “competitor RNA.”

Comparative RT-PCR resembles competitive RT-PCR in that target messages from each RNA sample compete for amplification reagents within a single reaction, making the technique reliably quantitative. Because the cDNA from both samples have the same PCR primer binding site, one sample acts as a competitor for the other, making it unnecessary to synthesize a competitor RNA sequence.

Both relative and competitive RT-PCR quantitation techniques may be combined with pilot experiments. In the case of relative RT-PCR, pilot experiments include selection of a quantitation method and determination of the exponential range of amplification for each mRNA under study. For competitive RT-PCR, a synthetic RNA competitor transcript must be synthesized and used in pilot experiments to determine the appropriate range for the standard curve. Comparative RT-PCR yields similar sensitivity as relative and competitive RT-PCR, but may require less optimization and does not require synthesis of a competitor.

Relative RT-PCR uses primers for an internal control that are multiplexed in the same RT-PCR reaction with the gene specific primers. In one embodiment internal control and gene specific primers are designed so that they do not produce additional bands or hybridize to each other. The expression of the internal control may be constant across all samples being analyzed so that the signal from the internal control can used to normalize sample data to account for differences caused by variable RNA quality or RT efficiency, inaccurate quantitation or pipetting variation. Common internal controls include, for example, GAPDH mRNAs and 18S rRNA.

Quantitative real-time RT-PCR is accurate, precise, high throughput, and relatively easy to perform. Real-time PCR automates the process of relative RT-PCR by quantitating reaction products for each sample in every cycle. The result is a broad dynamic range, with no user optimization required.

In some embodiments real-time PCR systems rely upon the detection and quantitation of a fluorescent reporter, the signal of which increases in direct proportion to the amount of PCR product in a reaction. In the simplest and most economical format, that reporter is the double-strand DNA-specific dye SYBR® Green (Molecular Probes). SYBR Green binds double-stranded DNA, and upon excitation emits light. Thus, as a PCR product accumulates, fluorescence increases.

Two alternatives to SYBR Green are TaqMan® and molecular beacons, both of which are hybridization probes relying on fluorescence resonance energy transfer (FRET) for quantitation.

TaqMan Probes are oligonucleotides that contain a fluorescent dye, typically on the 5′ base, and a quenching dye, typically located on the 3′ base. When irradiated, the excited fluorescent dye transfers energy to the nearby quenching dye molecule rather than fluorescing, resulting in a nonfluorescent substrate. TaqMan probes are designed to hybridize to an internal region of a PCR product. During PCR, when the polymerase replicates a template on which a TaqMan probe is bound, the 5′ exonuclease activity of the polymerase cleaves the probe. This separates the fluorescent and quenching dyes and FRET no longer occurs. Fluorescence increases in each cycle, proportional to the rate of probe cleavage.

Molecular beacons also contain fluorescent and quenching dyes, but FRET only occurs when the quenching dye is directly adjacent to the fluorescent dye. Molecular beacons are designed to adopt a haiipin structure while free in solution, bringing the fluorescent dye and quencher in close proximity. When a molecular beacon hybridizes to a target, the fluorescent dye and quencher are separated, FRET does not occur, and the fluorescent dye emits light upon irradiation.

Obtaining sufficient total RNA or mRNA for the study of gene expression using microarrays and also for validation using QRT-PCR is often problematic. Often only a small amount of RNA may be isolated from a sample and there is not enough RNA for the array analysis experiment as well as the validation experiment. For some samples it is necessary to use the entire amount of RNA isolated from the sample for amplification in order to obtain enough cRNA (copy RNA) for efficient hybridization. Validation of microarray results may be difficult for samples for which not enough total RNA is available. Validation is however important for data accuracy. Methods are disclosed for using cRNA for validation of array assays.

The amplified sample may be cDNA or cRNA and amplification may be by any method known in the art. For example, poly (A) RNA may be reverse transcribed using an oligo (dT)-T7 promoter primer and the resulting double stranded cDNA may be used to transcribe many copies of cRNA using T7 RNA polymerase. In another embodiment the poly (A) RNA or total RNA may be reverse transcribed using random primers and the resulting cDNA may be the amplified sample. Methods of amplification are disclosed, for example in U.S. Pat. No. 6,582,906, U.S. patent application Ser. Nos. 09/634,352, 10/763,414, 10/805,559, 10/090,320, 10/877,544 and 60/542,933 each of which is incorporated herein by reference in its entirety. cRNA may be derived by amplification of total RNA or mRNA. In one embodiment, total RNA is isolated from the sample and (polyA) mRNA is isolated from the total RNA. A cDNA copy of the mRNA is generated by reverse transcription using a primer that has a 5′ RNA polymerase promoter sequence, such as T7, and a 3′ stretch of T residues, for example 17-21 T's. The cDNA is then made into double stranded cDNA which has a functional RNA polymerase promoter. The ds-cDNA is then used as template for in vitro transcription using an RNA polymerase such as T7 polymerase. Multiple copies of the starting RNA are made. The copies are cRNA and in many embodiments the cRNA is labeled during synthesis (during the in vitro transcription step). The cRNA should be synthesized at levels that are directly proportional to the levels of the RNA in the starting sample. The labeled cRNA is fragmented and hybridized to an array of probes to generate an expression profile. The cRNA is an amplified representation of the starting material and is therefore much more abundant than the nucleic acids present in the starting sample. Other methods of amplification that may be used include, for example, multiple displacement amplification as described in Hosono et al., Genome Res. 13:954-964 (2003) and Dean et al. PNAS 99:5261-5266 (2002).

In one embodiment, cRNA is used as a starting material for RT-PCR validation or for QRT-PCR studies. Primers may be selected for the gene of interest using a primer design software program such as, PrimerExpress or Primer3. In one embodiment cRNA is labeled with biotin.

In one embodiment, cRNAs are hybridized to an array to detect differential gene expression. In another embodiment, quantitation of cRNAs is measured by quantitative RT-PCR. In one embodiment QRT-PCR is used to confirm gene expression differences observed by another method such as nucleic acid arrays.

In a preferred embodiment, total RNA is isolated from tissue or cells and reverse transcribed to produce cDNAs. Labeled cRNAs are then synthesized by in vitro transcription (IVT) from the cDNAs. In one embodiment, cRNAs may be fragmented before hybridization. See GeneChip Expression Analysis Technical Manual, available from Affymetrix, Inc. and incorporated herein by reference.

In a preferred embodiment, the total RNA is isolated from a given sample using, for example, an acid guanidinium-phenol-chloroform extraction method and poly(A) mRNA is isolated by oligo dT column chromatography or by using (dT)ₙ magnetic beads. (See e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987), which are incorporated by reference in their entireties for all purposes). (See also PCT/US99/25200 for complexity management and other sample preparation techniques, which is hereby incorporated by reference in its entirety for all purposes.) Where the single-stranded DNA population of the present invention is cDNA produced from a mRNA population, it may be produced according to methods known in the art. (See, e.g, Maniatis et al., supra, at 213-46). In a preferred embodiment, a sample population of single-stranded poly(A) RNA may be used to produce corresponding cDNA in the presence of reverse transcriptase, oligo-dT primer(s) and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be thermal stable (e.g., rTth DNA polymerase available from PE Applied Biosystems, Foster City, Calif.).

The RNA of the present invention may be obtained or derived from any tissue or cell source. Indeed, the nucleic acid sought to be amplified may be obtained from any biological or environmental source, including plant, virion, bacteria, fungi, or algae, from any sample, including body fluid or soil. In one embodiment, eukaryotic tissue is preferred, and in another, mammalian tissue is preferred, and in yet another, human tissue is preferred. The tissue or cell source may include a tissue biopsy sample, a cell sorted population, cell culture, an LCM sample, a fine needle aspirate or a single cell. In a preferred embodiment, the tissue source may include brain, liver, heart, kidney, lung, spleen, retina, bone, lymph node, endocrine gland, reproductive organ, blood, nerve, vascular tissue, and olfactory epithelium. In yet another preferred embodiment, the tissue or cell source may be embryonic or tumorigenic.

Tumorigenic tissue according to the present invention may include tissue associated with malignant and pre-neoplastic conditions, not limited to the following: acute lymphocytic leukemia, acute myelocytic leukemia, myeloblastic leukemia, promyelocytic leukemia, myelomonocytic leukemia, monocytic leukemia, erythroleukemia, chronic myelocytic (granulocytic) leukemia, chronic lymphocytic leukemia, polycythemia vera, lymphoma, Hodgkin's disease, non-Hodgkin's disease, multiple myeloma, Waldenstrom's macroglobulinemia, heavy chain disease, solid tumors, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. See Fishman et al., Medicine, 2d Ed. (J. B. Lippincott Co., Philadelphia, Pa. 1985), which is expressly incorporated herein by reference.

Example: cRNA vs total RNA

To examine the performance of the cRNA method, expression profiles obtained by the cRNA method were compared with those obtained by the total RNA method. AN3 CA (ATCC HTB-111) human endometrium adenocarcinoma cells were cultured and harvested according to standard methodology. Total RNA and poly (A) RNA were extracted. Total RNA was extracted using affinity resin (RNeasy, Qiagen, Chatsworth, Calif.). Poly(A) RNA was purified from total RNA using oligo-dT latex beads (Oligotex Direct, Qiagen). Double-stranded cDNA was synthesized from total RNA (Invitrogen Life Technologies SuperScript Choice system, Carlsbad, Calif.). In vitro transcription (IVT) was performed to generate cRNA (see Wodicka et al., Nat Biotechnol. 15:1359-1367, 1997, Mahadevappa and Warrington, Nature Biotechnol. 17:1134-1136, 1999). Comparison of transcript detection of cRNA and total RNA between 3 different samples (K7, K8, K10) was performed on four different genes: ribosomal RNA, GAPDH, nexin and calreticulin. A master mix of the reagents necessary to prepare labeled target was used in all RNA samples.

In order to compare the cRNA method, which uses polyA RNA as a starting point, to a method that uses total RNA as a starting point differences in the starting material should be taken into account to determine how much cRNA should be used to compare the results to the total RNA method. Four different possibilities were assumed i.e. that 1%, 2%, 5% or 10% of total RNA is poly(A) RNA. cRNA quantities that were equal to 1%, 2%, 5% or 10% of the total RNA quantity were used. For example, routinely 1 μg of total RNA is used for cDNA synthesis for QRT-PCR. Therefore 1 to 10% of 1 μg which is 10 to 100 ngs of cRNA was used and compared to the total RNA QRT-PCR results. Total RNA from an endometrial adenocarcinoma cell line ranging from 0.1 to 50 ng was used as a control. As expected the ribosomal RNA which is not polyadenylated was detected at very low levels in the cRNA sample. The results showed that the assumption of a range of 1% to 5% of cRNA yields results comparative to using total RNA for genes at various level of abundance. For example, for GAPDH for one of the samples the total RNA gave a result of 3.43 units and when the amount of cRNA used was equal to 5% of the amount of total RNA used the result was 3.11 units, indicating that an assumption that the polyA RNA was about 5% of the total RNA was reasonable.

1. A method for monitoring the expression of a plurality of genes in an experimental sample comprising:
(a) isolating total RNA from the experimental sample and isolating mRNA from the total RNA;
(b) amplifying the mRNA to obtain an amplified sample;
(c) removing a first aliquot of the amplified sample;
(d) hybridizing the remainder of the amplified sample to a microarray to obtain a hybridization pattern;
(e) analyzing the hybridization pattern to obtain a first expression measurement for each of a plurality of transcripts;
(e) analyzing the first aliquot of the amplified sample to obtain a second expression measurement for at least one selected mRNA species using a method selected from the following methods: quantitative reverse transcription-polymerase chain reaction (QRT-PCR), Northern blot hybridization, quantitative PCR, Southern blot hybridization and nuclease protection analysis; and
(f) comparing the second expression measurement to the first expression measurement obtained for said selected mRNA species to determine the variation between the first and second expression measurements wherein said variation is an indication of the accuracy of the first expression measurement.

2. The method of claim 1 wherein the mRNA is amplified by a method comprising:
isolating poly (A) RNA from said nucleic acid sample;
incubating the isolated poly (A) RNA with a primer comprising an RNA polymerase promoter sequence and a region of poly(dT) wherein the region of poly(dT) is 3′ of the RNA polymerase promoter sequence;
extending the primer using reverse transcriptase to generate first strand cDNA from said poly (A) RNA;
generating second strand cDNA resulting in double stranded cDNA comprising an RNA polymerase promoter; and
generating cRNA from the double stranded cDNA using an RNA polymerase that recognizes the RNA polymerase promoter.

3. The method of claim 2 wherein the RNA polymerase is T7 RNA polymerase.

4. The method of claim 2 wherein the cRNA is labeled.

5. The method of claim 4 wherein the cRNA is labeled with biotin.

6. The method of claim 2 further comprising amplifying the cRNA.

7. The method of claim 1 wherein the amount of nucleic acid analyzed in (e) is approximately equal to 1% of the amount of total RNA isolated from the experimental sample in step (a).

8. The method of claim 1 wherein the amount of nucleic acid analyzed in (e) is approximately equal to 2% of the amount of total RNA isolated from the experimental sample in step (a).

9. The method of claim 1 wherein the amount of nucleic acid analyzed in (e) is approximately equal to 5% of the amount of total RNA isolated from the experimental sample in step (a).

10. The method of claim 1 wherein the amount of nucleic acid analyzed in (e) is approximately equal to 10% of the amount of total RNA isolated from the experimental sample in step (a).

11. The method of claim 1 wherein the selected mRNA species is GAPDH.

12. The method of claim 1 wherein the selected mRNA species is Nexin.

13. The method of claim 1 wherein the mRNA is amplified by a method comprising: mixing the mRNA in a reaction comprising random primers and a reverse transcriptase to generate cDNA copies of said mRNA.

14. The method of claim 1 wherein said amplified sample is labeled by incorporation of a detectable label during amplification.

15. The method of claim 1 wherein said amplified sample is fragmented and end labeled after amplification.

16. The method of claim 1 wherein the experimental sample is obtained by laser capture microdissection.

17. The method of claim 1 wherein the experimental sample is a fine needle aspirate.
